(19) United States
(12) Patent Application Publication
(10) Pub. No.: US 2009/0253785 A1
(43) Pub. Date: Oct. 8, 2009

(54) Bactericide composition

(76) Inventor: Tai-Li Ou, Tainan (TW)

Correspondence Address: ANDREWS KURTH LLP, 1350 I STREET, N.W., SUITE 1100, WASHINGTON, DC, 20005, US

(21) Appl. No.: 11/655,836

(22) Filed: Jan. 22, 2007

(51) Int. Cl. A61K 31/34

(52) U.S. Cl. 514/474

(57) ABSTRACT

A bactericide composition kills Staphylococcus aureus (S. aureus), Escherichia coli (E. coli), Methicillin-resistant Staphylococcus aureus (MRSA) and Propionibacterium acnes (P. acnes) and comprises a solvent and L-ascorbic acid. The L-ascorbic acid is dissolved in the solvent in a range of 1 wt % to 40 wt %. Ascorbic acid (vitamin C) is water-soluble and is essential for people. Thus, ascorbic acid contacting people will not harm them, and if a person inhales ascorbic acid, ascorbic acid will be metabolized and excreted out of person's body. Therefore, the bactericide composition is safe and not harmful to a person's health.

### BACKGROUND OF THE INVENTION

1. Field of Invention The present invention relates to a bactericide composition, and more particularly to a bactericide composition that can kill bacteria but is not harmful to a person's health.

2. Description of the Related Art

Generally, when a person is injured and has an open wound, bacteria will infect the wound and cause inflammation and suppuration that may damage a person's organs or even cause them to die. Thus, a bactericide is usually used to keep the wound from becoming infected by bacteria.

Conventional bactericides, such as alcohol, tincture of iodine and hydrogen peroxide, can damage cytoplasm of bacteria to kill the bacteria, so that the wound will not be infected. However, most conventional bactericides are synthesized from chemicals, and the bactericide contacts a person's skin or he/she may inhale the bactericide, which damages a person's health.

To overcome the shortcomings, the present invention provides a bactericide composition to mitigate or obviate the aforementioned.

### SUMMARY OF THE INVENTION

The primary objective of the present invention is to provide a bactericide composition that can kill bacteria but is not harmful to a person's health.

To achieve the objective, the bactericide composition kills Staphylococcus aureus (S. aureus), Escherichia coli (E. coli), Methicillin-resistant Staphylococcus aureus (MRSA) and Propionibacterium acnes (P. acnes) and has an L-ascorbic acid solution. The L-ascorbic acid is dissolved in a solvent in a range of 1 wt % to 40 wt %. Ascorbic acid (vitamin C) is water-soluble and is essential for a person. Thus, ascorbic acid contacting a person's skin will not harm them, and if a person inhales ascorbic acid, the ascorbic acid will be metabolized and excreted from the person's body. Therefore, the bactericide in accordance with the present invention is safe and is not harmful to a person's health.

Other objectives, advantages and novel features of the invention will become more apparent from the following detailed description when taken in conjunction with the accompanying drawings.

### DETAILED DESCRIPTION OF THE INVENTION

A bactericide composition in accordance with the present invention comprises L-ascorbic acid and kills Staphylococcus aureus (S. aureus), Escherichia coli (E. coli), Methicillin-resistant Staphylococcus aureus (MRSA) and Propionibacterium acnes (P. acnes). The bactericide composition may be an ointment, solution, gel, spray or other forms and comprises a solvent and L-ascorbic acid. 1 wt % to 40 wt % L-ascorbic acid is dissolved in solvent. The solvent is selected from the group consisting of glycerol, N-methyl-2-pyrrolidone (NMP), alcohol, water and mixtures of glycerol, N-methyl-2-pyrrolidone (NMP), alcohol and water. The L-ascorbic acid solution is prepared by dissolving L-ascorbic acid in the selected solvent in a range of about 1 wt % to about 40 wt %. In a preferred embodiment, the L-ascorbic acid solution is prepared by dissolving L-ascorbic acid in the selected solvent in a range of about 15 wt % to about 40 wt % when the bactericide composition is used to kill S. aureus. In a preferred embodiment, the L-ascorbic acid solution is prepared by dissolving L-ascorbic acid in the selected solvent in a range of about 1 wt % to about 40 wt % when the bactericide is used to kill E. coli.

The bactericide composition can be applied to general skin injuries and injuries caused by lasers, atopic dermatitis, impetigo, acne or folliculitis, used to cleanse the large intestine, urinary tract or vagina or to preserve transplant organs, cosmetics or the like.

The following examples are provided to illustrate, but are not to be construed to limit the scope of, the invention.

The following examples are about a bactericide composition in accordance with the present invention that kills S. aureus, E. coli, MRSA and P. acnes.

1. Preparation of Bacterial Suspension:

S. aureus, E. coli, MRSA and P. acnes were respectively prepared to form a suspension. The concentration of the suspension was about 1.5×10⁸ CFU/ml (density=0.5 McFarland), and then was diluted 100 times (about 1.5×10⁶ CFU/ml).

2. Preparation of Bactericide Composition:

L-ascorbic acid solutions were prepared respectively in concentrations of 1 wt %, 5 wt %, 10 wt %, 15 wt %, 20 wt %, 25 wt % and 30 wt %. The different concentrations of L-ascorbic acid solutions were used as a test. A hydrochloric acid solution and distilled water were used respectively as a positive control and a negative control, and the pH value of the hydrochloric acid solution was the same of the pH value of the 25 wt % L-ascorbic acid solution.

3. Quantitative Test of the Bacterial Suspensions:

3.1

1 ml bacterial suspension was added to a 9 ml L-ascorbic acid solution for testing, and equal amounts of the bacterial suspension were added respectively to the positive control of 9 ml hydrochloric acid solution and the negative control of 9 ml distilled water, and then all solutions were incubated for 24 hours.

3.2

After incubating for 24 hours, all the solutions were series diluted, until diluted 1000 times to form a series of dilution solutions.

3.3

To calculate bacteria remaining in the solutions, 0.2 ml of each series of dilution solutions were cultured respectively on Petri dishes and repeated twice.

3.4

Each Petri dish was cultured in an incubator at 35±2° C. for 1˜2 days (S. aureus, E. coli) or for 2˜4 days (MRSA and P. acnes) to observe bacteria growth on Petri dish and the bacteria were counted.

4. Anti-Bacterial Ratio:

Anti-bacterial ratio (%)=[(remaining bacteria of controls−remaining bacteria of test)/remaining bacteria of controls]×100%

5. Results:

The results of experiments for S. aureus, E. coli, MRSA and R acnes are shown respectively in Table 1 to Table 4.

TABLE 1

Item

S. aureus

| | | Remaining | Anti-bacteria |
|---|---|---|---|
| | | bacteria | ratio |
| | pH | (CFU/mL) | (%) |
| Non-treatment | — | 5.6 × 10⁵ | — |
| Negative control | 7.75 | 8.9 × 10⁶ | — |
| (Distilled water) | | | |
| Positive control | 1.68 | 18 | 99.99 |
| (Hydrochloric acid) | | | |
| 1% L-ascorbic acid | 2.70 | 2.5 × 10⁴ | 99.72 |
| solution | | | |
| 5% | 2.28 | 3.0 × 10³ | 99.97 |
| 10% | 2.05 | 2.0 × 10² | 99.99 |
| 15% | 1.93 | 0 | 100 |
| 20% | 1.80 | 0 | 100 |
| 25% | 1.68 | 0 | 100 |
| 30% | 1.59 | 0 | 100 |

TABLE 2

Item

E. coli

| | | Remaining | Anti-bacteria |
|---|---|---|---|
| | | bacteria | ratio |
| | pH | (CFU/mL) | (%) |
| Non-treatment | — | 1.4 × 10⁵ | — |
| Negative control | 7.75 | 6.1 × 10⁶ | — |
| (Distilled water) | | | |
| Positive control | 1.68 | 0 | 100 |
| (Hydrochloric acid) | | | |
| 1% L-ascorbic acid | 2.70 | 0 | 100 |
| solution | | | |
| 5% | 2.28 | 0 | 100 |
| 10% | 2.05 | 0 | 100 |
| 15% | 1.93 | 0 | 100 |
| 20% | 1.80 | 0 | 100 |
| 25% | 1.68 | 0 | 100 |
| 30% | 1.59 | 0 | 100 |

TABLE 3

Item

MRSA

| | | Remaining | Anti-bacteria |
|---|---|---|---|
| | | bacteria | ratio |
| | pH | (CFU/mL) | (%) |
| Non-treatment | — | 8.9 × 10⁵ | — |
| Negative control | 7.75 | 4.0 × 10⁷ | — |
| (Distilled water) | | | |
| 1% L-ascorbic acid | 2.70 | 2.0 × 10³ | 99.99 |
| solution | | | |
| 5% | 2.28 | 0 | 100 |
| 10% | 2.05 | 0 | 100 |
| 15% | 1.93 | 0 | 100 |
| 20% | 1.80 | 0 | 100 |
| 25% | 1.68 | 0 | 100 |
| 30% | 1.59 | 0 | 100 |

TABLE 4

Item

P. acnes

| | | Remaining | Anti-bacteria |
|---|---|---|---|
| | | bacteria | ratio |
| | pH | (CFU/mL) | (%) |
| Non-treatment | — | 1.8 × 10⁵ | — |
| Negative control | 7.75 | 1.4 × 10⁵ | — |
| (Distilled water) | | | |
| 1% L-ascorbic acid | 2.70 | 0 | 100 |
| solution | | | |
| 5% | 2.28 | 0 | 100 |
| 10% | 2.05 | 0 | 100 |
| 15% | 1.93 | 0 | 100 |
| 20% | 1.80 | 0 | 100 |
| 25% | 1.68 | 0 | 100 |
| 30% | 1.59 | 0 | 100 |

According to Table 1 to Table 4, original concentrations of S. aureus, E. coli, MRSA and P. acnes were respectively 5.6×10⁵, 1.4×10⁵, 8.9×10⁵, 1.8×10⁵ CFU/ml. After adding different concentrations of L-ascorbic acid solutions and incubating for 24 hours with S. aureus, E. coli, MRSA and P. acnes, no remaining E. coli and P. acnes were found in the Petri dishes with concentration of 1 wt % to 30 wt % L-ascorbic acid solution, no remaining S. aureus were found with the concentrations of 15 wt % to 30 wt % L-ascorbic acid solution and no remaining MRSA were found with the concentration of 5 wt % to 30 wt %.

Consequently, the bactericide composition containing ascorbic acid (vitamin C) with a concentration of more than 1 wt % displays a bactericidal property. Further, ascorbic acid is an essential vitamin to humans so ascorbic acid contacting people will not harm them Therefore, the bactericide composition in accordance with the present invention is safe and is not harmful to a person's health.

Even though numerous characteristics and advantages of the present invention have been set forth in the foregoing description, together with details of the structure and function of the invention, the disclosure is illustrative only. Changes may be made in detail, especially in matters of shape, size and arrangement of parts within the principles of the invention to the full extent indicated by the broad general meaning of the terms in which the appended claims are expressed.

What is claimed is:

1. A bactericide composition, comprising
a solvent, and
1 wt % to 40 wt % L-ascorbic acid dissolved in the solvent.

2. The bactericide composition as claimed in claim 1, wherein the bactericide composition is an ointment.

3. The bactericide composition as claimed in claim 1, wherein the bactericide composition is a solution.

4. The bactericide composition as claimed in claim 1, wherein the bactericide composition is a gel.

5. The bactericide composition as claimed in claim 1, wherein the bactericide composition is a spray.

6. The bactericide composition as claimed in claim 1, which kills S. aureus, and the L-ascorbic acid is in a range of 15 wt % to 40 wt %.

7. The bactericide composition as claimed in claim 1, which kills E. coli, and the L-ascorbic acid is in a range of 1 wt % to 40 wt %.

8. The bactericide composition as claimed in claim 3, wherein the solvent is selected from the group of glycerol, N-methyl-2-pyrrolidone (NMP), alcohol, water and mixtures of glycerol, N-methyl-2-pyrrolidone (NMP), alcohol and water.

9. The bactericide composition as claimed in claim 6, wherein the L-ascorbic acid is in a range of 15 wt % to 30 wt %.

10. The bactericide composition as claimed in claim 7 which kills E. coli, and the L-ascorbic acid is in a range of 1 wt % to 30 wt %.

11. A method for killing bacteria comprising killing bacteria with a bactericide composition composed of
a solvent, and
1 wt % to 40 wt % L-ascorbic acid dissolved in the solvent.

12. The method as claimed in claim 11, wherein the bacteria is selected from the group consisting of Staphylococcus aureus (S. aureus), Escherichia coli (E. coli), Methicillin-resistant Staphylococcus aureus (MRSA) and Propionibacterium acnes (P. acnes).

13. The bactericide composition as claimed in claim 11, wherein the bactericide composition is an ointment.

14. The method as claimed in claim 11, wherein the bactericide composition is a solution.

15. The method as claimed in claim 11, wherein the bactericide composition is a gel.

16. The method as claimed in claim 11, wherein the bactericide composition is a spray.

17. The method as claimed in claim 14, wherein the solvent is selected from the group of glycerol, N-methyl-2-pyrrolidone (NMP), alcohol, water and mixtures of glycerol, N-methyl-2-pyrrolidone (NMP), alcohol and water.

18. The method as claimed in claim 11, which kills S. aureus, and the L-ascorbic acid is in a range of 15 wt % to 40 wt %.

19. The method composition as claimed in claim 11, which kills E. coli, and the L-ascorbic acid is in a range of 1 wt % to 40 wt %.
